(19) United States
(12) Patent Application Publication
(10) Pub. No.: US 2005/0142489 A1
(43) Pub. Date: Jun. 30, 2005

(54) Squarylium dyes as light-absorbing compound in the information layer of optical data carriers

(76) Inventors: Horst Berneth, Leverkusen (DE); Friedrich-Karl Bruder, Krefeld (DE); Wilfried Haese, Odenthal (DE); Karin Hassenruck, Dusseldorf (DE); Serguei Kostromine, Swisstal (DE); Peter Landenberger, Koln (DE); Thomas Sommermann, Bergish Gladbach (DE); Josef-Walter Stawitz, Odenthal (DE); Rainer Hagen, Leverkusen (DE); Rafael Oser, Krefeld (DE); Christa-Maria Kruger, Munster (DE); Timo Meyer-Friedrichsen, Krefeld (DE)

Correspondence Address: Lanxess Corporation;Law & Intellectual Property Department, 100 Bayer Road, Pittsburgh, PA, 15205-9741, US

(21) Appl. No.: 10/508,417

(22) PCT Filed: Mar. 18, 2003
(86) PCT No.: PCT/EP03/02789

(30) Foreign Application Priority Data
Mar. 19, 2002 (DE) 102 12 199.0
Sep. 27, 2002 (DE) 102 45 581.3

(51) Int. Cl. C09B 47/04; C09B 62/00; C09B 67/00; C07D 43/02; G11B 7/24

(52) U.S. Cl. 430/270.15; 430/270.16; 548/517; 540/128

(57) ABSTRACT

Novel squarylium compounds for optical data carriers have been found. The latter comprise a preferably transparent substrate to whose surface a light-writable information layer, if desired one or more reflection layers and a further substrate or a protective layer have been applied and can be written on and read by means of red light, preferably laser light, where the information layer comprises a light-absorbent compound and, if desired, a binder. They are characterized in that at least the said squarylium compounds are used as light-absorbent compound.

The invention relates to squarylium dyes, to a process for preparing them, to the components on which the squarylium dyes are based and their preparation, and to optical data carriers comprising the squarylium dyes in their information layer.

Write-once optical data carriers using specific light-absorbent substances or mixtures thereof are particularly suitable for use in DVD-R disks which operate with red (635-660 nm) laser diodes and for the application of the abovementioned dyes to a polymer substrate, in particular polycarbonate, by spin coating.

The write-once compact disk (CD-R, 780 nm) has recently experienced enormous volume growth and represents the technically established system.

The next generation of optical data stores—DVDs—is currently being introduced onto the market. The use of shorter-wavelength laser radiation (635-660 nm) and higher numerical aperture NA enables the storage density to be increased. The writable format in this case is DVD-R.

The achievable storage density depends on the focusing of the laser spot on the information plane. Spot size scales with the laser wavelength λ/NA. NA is the numerical aperture of the objective lens used. In order to obtain the highest possible storage density, the use of the smallest possible wavelength λ is the aim. At present, 390 nm is possible on the basis of semiconductor laser diodes.

Apart from the abovementioned optical properties, the writable information layer comprising light-absorbent organic substances has to have a substantially amorphous morphology to keep the noise signal during writing or reading as small as possible. For this reason, it is particularly preferred that crystallization of the light-absorbent substances be prevented in the application of the substances by spin coating from a solution, by vapour deposition and/or sublimation during subsequent coating with metallic or dielectric layers under reduced pressure.

The amorphous layer comprising light-absorbent substances preferably has a high heat distortion resistance, since otherwise further layers of organic or inorganic material which are applied by sputtering or vapour deposition can form blurred boundaries due to diffusion and thus adversely affect the reflectivity. Furthermore, a light-absorbent substance which has insufficient heat distortion resistance can, at the boundary to a polymeric support, diffuse into the latter and once again adversely affect the reflectivity.

A light-absorbent substance whose vapour pressure is too high can sublime during the abovementioned deposition of further layers by sputtering or vapour deposition in a high vacuum and thus reduce the desired layer thickness. This in turn has an adverse effect on the reflectivity.

It is therefore an object of the invention to provide suitable compounds which satisfy the high requirements (e.g. light stability, favourable signal/noise ratio, damage-free application to the substrate material, and the like) for use in the information layer in a write-once optical data carrier for writable optical data store formats in a laser wavelength range from 600 to 680 nm.

It has surprisingly been found that light-absorbent compounds selected from the group of specific symmetrical squarylium compounds can satisfy the abovementioned requirement profile particularly well.

The invention accordingly provides squarylium compounds of the general formula I, 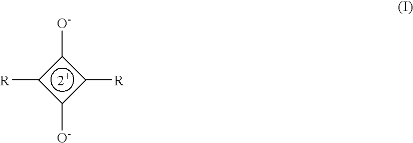
where
R is a heterocyclic five-membered ring, in particular a substituted or unsubstituted pyrrole, with the exception of amino-substituted furan rings.

Preference is given to squarylium compounds of the formula I which correspond to the formula Ia, 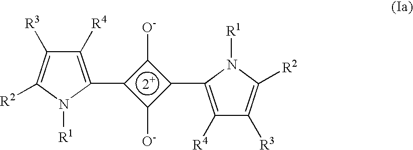
where
R¹ is hydrogen, substituted or unsubstituted alkyl or substituted or unsubstituted aralkyl,
R² is substituted or unsubstituted alkyl, substituted or unsubstituted aryl, alkoxycarbonyl or substituted or unsubstituted alkylcarbonyl, and
R³ and R⁴ are each, independently of one another, hydrogen, substituted or unsubstituted alkyl, substituted or unsubstituted aryl, alkoxycarbonyl or substituted or unsubstituted alkylcarbonyl.

Formula Ia is one of the possible mesomeric formulae.

For the purposes of the present application, “alkyl” is preferably C₁-C₆-alkyl, “aryl” is preferably C₆-C₁₀-aryl, “aralkyl” is preferably C₇-C₁₆-aralkyl and “alkoxy” is preferably C₁-C₆-alkoxy.

Possible substituents on the alkyl, aryl or aralkyl radicals are halogen, in particular F, hydroxy, nitro, cyano, carboxyl, alkoxy, trialkylsilyl and trialkylsiloxy. The alkyl radicals can be linear, cyclic or branched. They can be partially halogenated or perhalogenated. Examples of substituted alkyl radicals are trifluoromethyl, chloroethyl, cyanoethyl, methoxyethyl. Examples of cyclic alkyl radicals are cyclohexylmethyl and cyclopropylmethyl. Examples of branched alkyl radicals are isopropyl, tert-butyl, 2-butyl, neopentyl. Examples of possible aryl radicals are phenyl, 4-methoxyphenyl, 4-cyanophenyl, 3,5-bis(trifluoromethyl)phenyl, 4-trifluoromethylphenyl and 4-ethylphenyl. Examples of aralkyl radicals are benzyl, phenethyl, phenylpropyl, 4-methoxybenzyl, 4-cyanobenzyl, 3,5-bis(trifluoromethyl)benzyl, 4-trifluoromethylbenzyl and 4-ethylbenzyl. Examples of carboxyl radicals are ethoxycarbonyl, butoxycarbonyl and trifluoromethoxycarbonyl.

Examples of alkylcarbonyl are acetyl, trifluoroacetyl, propanoyl, butanoyl, pentanoyl and hexanoyl.

Preferred substituted or unsubstituted alkyl radicals are methyl, ethyl, n-propyl, n-pentyl, isobutyl, isopropyl, perfluorinated methyl and ethyl.

Preferred substituted or unsubstituted aralkyl radicals are, for example, 4-trifluoromethylbenzyl, 2-trifluoromethylbenzyl, 3,5-bistrifluoromethylbenzyl and 4-fluoro-2-trifluoromethylbenzyl.

A preferred alkoxycarboxyl radical is ethoxycarbonyl.

Particular preference is given to squarylium compounds of the formula Ia in which
R¹ is hydrogen, methyl, ethyl, propyl, butyl, cyclohexylmethyl, benzyl, 4-methoxybenzyl, 4-trifluoromethylbenzyl, 3-trifluoromethylbenzyl, 2-trifluoromethylbenzyl, 3,5-bis(trifluoromethyl)benzyl or 4-fluoro-2-trifluoromethylbenzyl,
R² is methyl, ethyl, propyl or phenyl and
R³ and R⁴ are each, independently of one another, hydrogen, methyl, ethyl, propyl, butyl, phenyl, acetyl or ethoxycarbonyl.

Even greater preference is given to squarylium compounds of the formula Ia in which
R¹ is 4-trifluoromethylbenzyl or 3,5-bis(trifluoromethyl)benzyl, in particular 4-trifluoromethylbenzyl,
R² is methyl, ethyl or phenyl, in particular methyl,
R³ is hydrogen, ethyl, acetyl or ethoxycarbonyl, in particular ethyl, and
R⁴ is methyl, ethyl or phenyl, in particular methyl or ethyl.

The invention further provides a process for preparing the squarylium compounds of the invention, which is characterized in that 3,4-dihydroxy-3-cyclobutene-1,2-dione (squaric acid) is reacted with at least one compound of the formula III
R—R⁵ (III),
in particular with a pyrrole compound of the formula (IIIa), preferably in a suitable solvent, 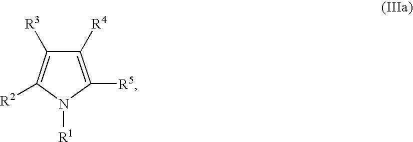
where
R⁵ is hydrogen, alkoxycarbonyl, in particular t-butoxycarbonyl or ethoxycarbonyl, or carboxyl and
R and R¹ to R⁴ are as defined above.

The process of the invention is preferably carried out in alcohol, in particular in ethanol. Preferred reaction temperatures are greater than 70° C., in particular 75-85° C. The process of the invention is likewise preferably carried out in aqueous acetic acid. The mixing ratio of acetic acid/water is, for example, from 3:1 to 1:3, preferably from 2:1 to 1:2, particularly preferably 1:1. Preferred reaction temperatures range from room temperature to the boiling point of the medium. Preference is likewise given to using a catalytic amount of a mineral acid, in particular HCl. The product generally precipitates as a pure solid from the reaction solution and is preferably washed with ether after being separated off.

The pyrrole compounds of the formula (IIIa) which are preferably used for preparing the squarylium compounds of the invention are likewise provided by the present invention.

The invention therefore also provides pyrroles of the formula (IIIa) 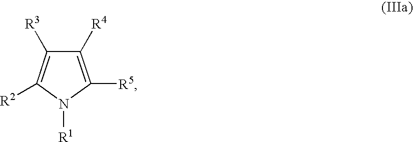
where
R¹ is substituted or unsubstituted C₃-C₁₂-alkyl or substituted or unsubstituted aralkyl,
R² is substituted or unsubstituted alkyl, substituted or unsubstituted aryl, alkoxycarbonyl, carbonyl or substituted or unsubstituted alkylcarbonyl,
R³ and R⁴ are each, independently of one another, hydrogen, substituted or unsubstituted alkyl, substituted or unsubstituted aryl, alkoxycarbonyl, carbonyl or substituted or unsubstituted alkoxycarbonyl and
R⁵ is hydrogen, alkoxycarbonyl or carboxyl.

Particular preference is given to pyrrole compounds of the formula IIIa in which
R¹ is propyl, butyl, cyclohexylmethyl, benzyl, 4-methoxybenzyl, 4-trifluoromethylbenzyl, 3-trifluoromethylbenzyl, 2-trifluoromethylbenzyl, 3,5-bis(trifluoromethyl)benzyl or 4-fluoro-2-trifluoromethylbenzyl,
R² is methyl, ethyl, propyl or phenyl,
R³ and R⁴ are each, independently of one another, hydrogen, methyl, ethyl, propyl, butyl, phenyl, acetyl or ethoxycarbonyl and
R⁵ is hydrogen, t-butoxycarbonyl or carboxyl.

The invention likewise provides a process for preparing the novel pyrrole compounds of the formula IIIa, which is characterized in that a pyrrole compound of the formula (II) 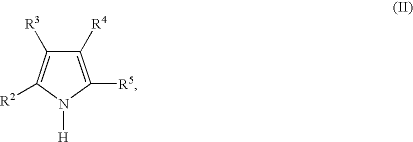
where
R² to R⁴ are as defined above for the novel pyrroles of the formula IIIa and
R⁵ is hydrogen, alkoxycarbonyl, in particular t-butoxycarbonyl, ethoxycarbonyl, or carboxyl,
   are reacted with a halogen compound of the formula IV
   R¹—X (IV),
   where
R¹ is as defined for the novel pyrroles of the formula IIIa,
X is Cl, Br or I,
   and at least 2 equivalents of a base.

A particularly suitable base is KOH. The reaction is preferably carried out in a suitable solvent, for example in dimethyl sulphoxide (DMSO), dimethylformarnmide (DMF) or a mixture thereof. The reaction is preferably carried out at a temperature of 20-100° C., particular preferably 50-90° C., in particular 65-80° C. If R⁵ in formula II is ethoxycarbonyl, the ester group is saponified to give the corresponding alkali metal salt of the pyrrolecarboxylic acid. This can be precipitated from aqueous solution by acidification and filtered off.

The product can in this way be obtained in satisfactory purity without complicated crystallization steps or similar purification steps.

The invention further provides for the use of the squarylium dyes of the invention as light-absorbent compounds in the information layer of write-once optical data carriers.

In this use, the optical data carrier is preferably written on and read by means of red laser light, in particular laser light having a wavelength in the range 600-680 nm.

The invention further provides for the use of squarylium compounds as light-absorbent compounds in the information layer of write-once optical data carriers which can be written on and read by means of red laser light, in particular laser light having a wavelength in the range 600-680 nm.

The invention further provides an optical data carrier comprising a preferably transparent substrate to whose surface a light-writable information layer, if desired one or more reflection layers and a further substrate or a protective layer have been applied, which can be written on and read by means of red light, preferably having a wavelength in the range 600-680 nm, preferably laser light, where the information layer comprises a light-absorbent compound and, if desired, a binder, characterised in that at least one squarylium dye according to the invention is used as light-absorbent compound.

The light-absorbent compound should preferably be able to be changed thermally. The thermal change preferably occurs at a temperature of <600° C., particularly preferably at a temperature of <400° C., very particularly preferably at a temperature of <300° C., in particular <200° C. Such a change can be, for example, a decomposition or chemical change of the chromophoric centre of the light-absorbent compound.

It is likewise preferred that the light-absorbent compound can be changed thermally only at above 100° C.

Preferred embodiments of the light-absorbent compounds in the optical data stores of the invention correspond to the preferred embodiments of the squarylium dye of the invention.

In a preferred embodiment, the light-absorbent compounds used are compounds of the formula (Ia) in which
R¹ is hydrogen, methyl, ethyl, propyl, butyl, cyclohexylmethyl, benzyl, 4-methoxybenzyl, 4-trifluoromethylbenzyl, 3-trifluoromethylbenzyl or 3,5-bis(trifluoromethyl)benzyl,
R², R³ and R⁴ are each, independently of one another, hydrogen, methyl, ethyl, propyl, butyl, phenyl, acetyl or ethoxycarbonyl.

In a particularly preferred embodiment, the light-absorbent compounds used are compounds of the formula (Ia) in which
R¹ is 4-trifluoromethylbenzyl or 3,5-bis(trifluoromethyl)benzyl or, in particular, 4-trifluoromethylbenzyl,
R² and R⁴ are each, independently of one another, methyl, ethyl or phenyl, in particular methyl or ethyl, and
R³ is hydrogen, ethyl, acetyl or ethoxycarbonyl, in particular ethyl.

In the case of the write-once optical data carrier of the invention which is written on and read by means of the light of a red laser, preference is given to light-absorbent compounds whose absorption maximum λₘₐₓ is in the range from 500 to 650 mm, where the wavelength λ_{1/2} at which the absorbance in the long wavelength flank of the absorption maximum at the wavelength λₘₐₓ₂ is half of the absorbance value at λₘₐₓ and the wavelength λ_{1/10} at which the absorbance in the long wavelength flank of the absorption maximum at the wavelength λₘₐₓ is one tenth of the absorbance value at λₘₐₓ are preferably not more than 60 nm apart. Such a light-absorbent compound preferably has no longer-wavelength maximum λₘₐₓ₃ up to a wavelength of 750 nm, particularly preferably 800 nm, very particularly preferably 850 nm.

Preference is given to light-absorbent compounds having an absorption maximum λₘₐₓ of from 510 to 620 nm.

Particular preference is given to light-absorbent compounds having an absorption maximum λₘₐₓ of from 530 to 610 nm.

Very particular preference is given to light-absorbent compounds having an absorption maximum λₘₐₓ of from 550 to 600 nm.

In these light-absorbent compounds, λ_{1/2} and λ_{1/10} as defined above, are preferably not more than 50 nm apart, particularly preferably not more than 40 nm apart, very particularly preferably not more than 30 nm apart.

The light-absorbent compounds preferably have a molar extinction coefficient ε of >60000 l/mol cm, more preferably >80000 l/mol cm, particularly preferably >100000 l/mol cm, very particularly preferably >120000 l/mol cm.

Particularly suitable squarylium compounds are those in which the dipole moment change Δμ=|μ_{g}−μ_{ag}|, i.e. the positive difference between the dipole moments in the ground state and in the first excited state, is very small, preferably <5 D, particularly preferably <2 D. A method of determining such a dipole moment change A1 is described, for example, in F. Würthner et al., Angew. Chem. 1997, 109, 2933, and in the literature cited therein. A low solvent-induced wavelength shift (dioxane/DMF) is likewise a suitable selection criterion. Preference is given to squarylium compounds whose solvent-induced wavelength shift Δλ=|λ_{DMF}−λ_{dioxane}|, i.e. the positive difference between the absorption wavelengths in the solvents dimethylformamide and dioxane, is <20 nm, particularly preferably <10 run, very particularly preferably <5 nm.

The absorption spectra are preferably measured in solution.

The light-absorbent compounds used according to the invention preferably make it possible to achieve a reflectivity of >10% in the optical data carrier in the unwritten state and a sufficiently high absorption for thermal degradation of the information layer on point-wise illumination with focused light if the wavelength of the light is in the range from 600 to 680 nm. The contrast between written and unwritten points on the data carrier is achieved by the reflectivity change of the amplitude and also the phase of the incident light due to the changed optical properties of the information layer after the thermal degradation.

The squarylium dyes of the invention are preferably applied to the optical data carrier by spin coating. They can be mixed with one another or with other dyes having similar spectral properties. The information layer can comprise not only the squarylium dyes of the invention but also additives such as binders, wetting agents, stabilizers, diluents and sensitizers and also further constituents.

Apart from the information layer, further layers such as metal layers, dielectric layers and protective layers may also be present in the optical data store of the invention. Metals and dielectric layers serve, inter alia, to adjust the reflectivity and the heat absorption/retention. Metals can be, depending on the laser wavelength, gold, silver, aluminium, etc. Examples of dielectric layers are silicon dioxide and silicon nitride. Protective layers are, for example, photocurable surface coatings, (pressure-sensitive) adhesive layers and protective films.

Pressure-sensitive adhesive layers consist mainly of acrylic adhesives. Nitto Denko DA-8320 or DA-8310, disclosed in the patent JP-A 11-273147, can, for example, be used for this purpose.

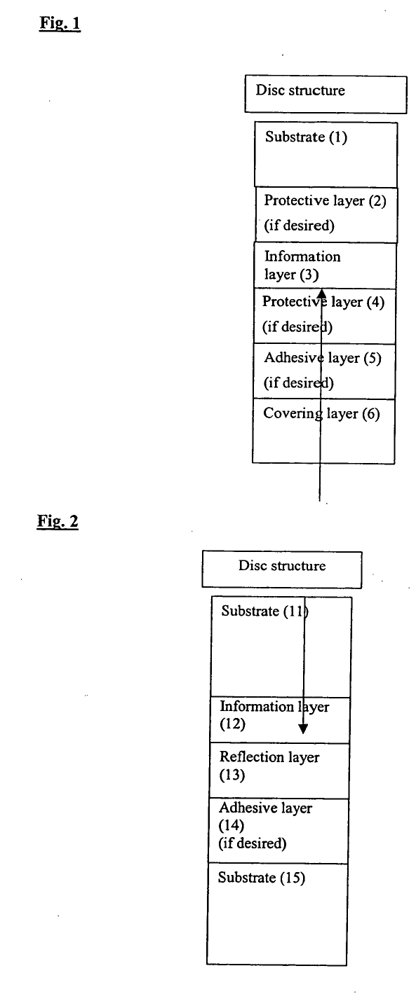

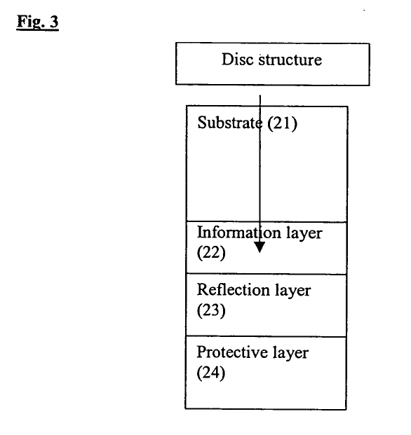

The optical data carrier of the invention has, for example, the following layer structure (cf. FIG. 2): a preferably transparent substrate (11), an information layer (12), if desired a reflection layer (13), if desired an adhesive layer (14), a further preferably transparent substrate (15).

In FIG. 2, the substrate (15) is preferably replaced by a sequence of layers (13), (12) and (11).

Alternatively, the structure of the optical data carrier can
comprise a plurality of information layers which are preferably separated by suitable layers. Particularly preferred separating layers are photocurable surface coatings, adhesive layers or reflection layers.

The arrows shown in FIG. 1, FIG. 2 and FIG. 3 indicate the path of the incident light.

The invention further provides optical data carriers according to the invention which have been written on by means of red light, in particular red laser light, particularly preferably having a wavelength of 600-680 nm.

The invention likewise provides a write-once optical data carrier whose information layer comprises at least one phthalocyanine dye as light-absorbent compound, and also provides a process for producing it.

It is accordingly an object of the invention to provide suitable compounds which satisfy the high requirements (e.g. light stability, favourable signal/noise ratio, damage-free application to the substrate material, and the like) for use in the information layer in a write-once optical data carrier, in particular for high-density writable optical data store formats, in a laser wavelength range from 360 to 460 nm.

It has surprisingly been found that specific phthalocyanines as light-absorbent compounds can satisfy the abovementioned requirement profile particularly well.

Phthalocyanines display an intense absorption in the wavelength range 360-460 nm which is important for lasers, namely the B or Soret bands.

The present invention accordingly provides an optical data carrier comprising a preferably transparent substrate which may, if desired, have previously been coated with one or more reflective layers and to whose surface a light-writable information layer, if desired one or more reflection layers and if desired a protective layer or a further substrate or a covering layer have been applied, which can be written or read by means of blue light, preferably laser light, particularly preferably light having a wavelength of 360-460 nm, in particular 380-420 nm, very particularly preferably 390-410 nm, or by means of infrared light, preferably laser light, particularly preferably light having a wavelength of 760-830 nm, where the information layer comprises a light-absorbent compound and, if desired, a binder, characterized in that at least one phthalocyanine of the formula (I) 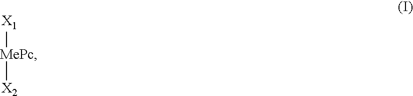
where
Me is a doubly axially substituted metal atom from the group consisting of Si, Ge and Sn,
Pc is an unsubstituted phthalocyanine and
X¹ and X² are each, independently of one another, bromine or iodine and X¹ may also be chlorine,
   is used as light-absorbent compound.

Preference is given to phthalocyanines of the formulae (Ia), (Ib), (Ic), (Id), (Ie), (If), (Ig) and (Ih)

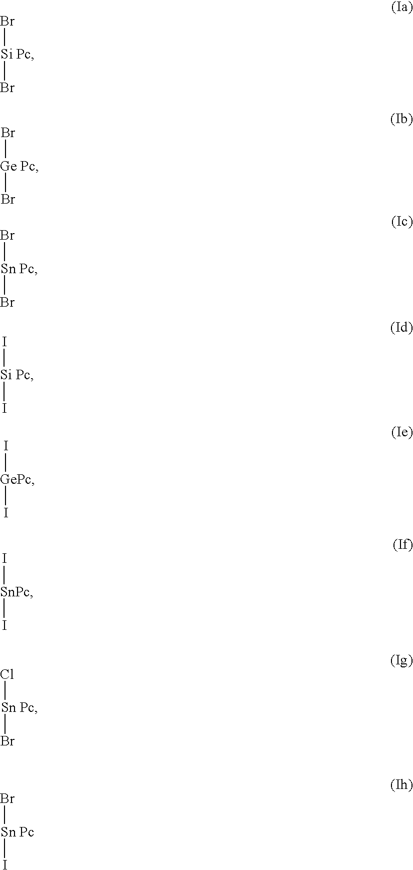

The formulae (Ic) and (If) to (Ih) are to be interpreted as indicating that the two halogen atoms are either both located above the plane of the phthalocyanine ring or one is located above and one is located below the plane of the phthalocyanine ring.

The phthalocyanines used according to the invention are known from, for example, J. N. Esposito, L. E. Sutton, M. E. Kenney, Inorg. Chem. 6, 1967, 1116 and W. J. Kroenke, M. E. Kenney, Inorg. Chem. 3, 1964, 696, or can be prepared as described there.

They can in principle be prepared by known methods, e.g.:
by ring synthesis from phthalonitrile or amino-imino-isoindole in the presence of the appropriate metal halides,
if desired, reaction of the products with water in suitable solvents, for example pyridine, to form phthalocyanines of the formula (I) in which X¹=X²=OH,
if desired, replacement of the axial substituents X¹=X²=halide by other appropriate halides,
if desired, replacement of the axial substituents X¹=X²=OH by appropriate halides by reaction with HX¹/HX²,
if desired, by oxidation of a nonaxially substituted phthalocyanine of the formula (II)
   MePc (II),
   by means of bromine, iodine, bromine chloride or iodine bromide.

The phthalocyanines of the formula (II), preferably those in which Me=Sn, are prepared, for example, by reduction of phthalocyanines of the formula (I), preferably with Me=Sn, in which X¹ and X² are halogen, for example chlorine. A suitable reducing agent is, for example, sodium tetrahydroborate (NaBH₄).

The light-absorbent compounds can be changed thermally. The thermal change preferably occurs at a temperature of <600° C. Such a change can be, for example, a decomposition or chemical change of the chromophoric centre of the light-absorbent compound.

The light-absorbent substances described guarantee a sufficiently high reflectivity of the optical data carrier in the unwritten state and a sufficiently high absorption for thermal degradation of the information layer on point-wise illumination with focused blue light, in particular laser light, preferably having a wavelength in the range from 360 to 460 nm. The contrast between written and unwritten points on the data carrier is achieved by the reflectivity change of the amplitude and also the phase of the incident light due to the changed optical properties of the information layer after the thermal degradation.

This means that the optical data carrier can preferably be written on and read by means of laser light having a wavelength of 360-460 nm.

The optical data carrier can likewise be written on and read by means of infrared light, in particular laser light having a wavelength of 760-830 nm, with the groove spacing and groove geometry then preferably being matched to the wavelength and numerical aperture.

The invention further provides for the use of the phthalocyanines of the formula (I) as light-absorbent compounds in the information layer of optical storage media.

The invention likewise provides for the use of the phthalocyanines of the formula (I) for producing optical storage media. The phthalocyanines are preferably used as light-absorbent compounds in the information layer.

The phthalocyanines which are particularly preferably employed in these uses have a content of more than 90% by weight, in particular more than 95% by weight, particularly preferably more than 98% by weight, based on the phthalocyanine of the formula (I).

The invention further provides a particulate solid preparation of a phthalocyanine of the formula (I), characterized in that the particles have a mean particle size of from 0.5 μm to 10 mm.

In a preferred embodiment of the particulate solid preparations, preference is given to those which have a mean particle size of from 0.5 to 20 μm, in particular from 1 to 10 μm, hereinafter referred to as fine powder. Such fine powders can be produced, for example, by milling.

Preference is likewise given to particulate solid preparations having a mean particle size of from 50 to 300 μm, hereinafter referred to as the finely crystalline form.

Further preferred particulate solid preparations are ones which have a mean particle size of from 50 μm to 10 mm, preferably from 100 μm to 800 μm, and form a particulate shaped body as agglomerates or conglomerates of primary particles. Such shaped bodies can, for example, have the shape of droplets, raspberries, flakes or rods, hereinafter referred to as granular materials.

The particle size of the finely crystalline form can, for example, be set via the parameters in the synthesis. For example, rapid heating, for example over a period of from 30 to 60 minutes, of the mixture of the components (phthalonitrile or aminoimino-isoindole and the appropriate metal halide in the appropriate solvent) to the reaction temperature, for example from 160 to 220° C., preferentially forms a finely divided form. A similar result is obtained when the metal halide is added to the reaction mixture (phthalonitrile or amino-imino-isoindole in the appropriate solvent) only at the reaction temperature, for example at from 160 to 190° C. Slow heating, for example over a period of from 65 to 250 minutes, of the mixture of the components to the reaction temperature, for example from 160 to 220° C., preferentially forms a coarse form.

The particulate solid preparations of the invention preferably comprise
80-100% by weight, preferably 95-100% by weight, of phthalocyanine of the formula (I),
0.1-1.0% by weight, preferably 0.1-0.5% by weight, of residual moisture,
0-10% by weight of inorganic salts,
0-10% by weight, preferably 0-5% by weight, of further additives such as dispersants, surfactants and/or wetting agents,
   where the percentages are in each case based on the preparation and the sum of the proportion indicated is 100%.

The solid preparations of the invention are preferably low in dust, free-flowing and have a good storage stability.

The granular materials can be produced in various ways, e.g. by spray drying, fluidized-bed spray granulation, fluidized-bed buildup granulation or powder fluidized-bed agglomeration.

Preference is given to granulation by spray drying, with both rotary disc analyzers and single-fluid or two-fluid nozzles, inter alia, being possible as spraying device. Preference is given to the single-fluid nozzle, in particular the swirl chamber nozzle, which is preferably operated at a feed pressure of 20-80 bar.

The inlet and outlet temperatures during spray drying depend on the desired residual moisture content, on safety measures and on economic considerations. The inlet temperature is preferably 120-200° C., in particular 140-180° C., and the outlet temperature is preferably 40-80° C.

The granular materials are generally produced by firstly mixing the dye filter cake, if appropriate together with auxiliaries and additives, intensively in a stirred vessel. The crystals of the suspension are preferably comminuted in a mill, e.g. a bead mill, so that a finely divided atomizable suspension is obtained.

In a preferred embodiment, the dye suspension is an aqueous suspension. Granulation is carried out by spray drying.

The invention further provides solid shaped bodies such as pellets, extrudates, etc., comprising a phthalocyanine of the formula (I), preferably in an amount of more than 90% by weight, in particular more than 95% by weight, preferably more than 98% by weight, based on the shaped body. Further additives to the solid shaped bodies can be binders. The sum of phthalocyanine of the formula (I) and binder is preferably more than 95% by weight, particularly preferably more than 99% by weight.

Such shaped bodies can be produced, for example, by pressing the phthalocyanine of the formula (I), if desired in the presence of binders, at a pressure of from 5 to 50 bar, preferably from 10 to 20 bar.

The invention likewise provides dispersions, preferably aqueous dispersions, containing a metal complex of the formula (I), preferably in an amount of from 10 to 90% by weight, based on the dispersion. Possible dispersants are, for example: polymeric dispersants based on acrylates, urethanes or long-chain polyoxyethylene compounds. Examples of suitable products are: Solsperse 32000 or Solsperse 38000 from Avecia.

The invention further provides a process for coating substrates with the phthalocyanines of the formula (1). This is preferably carried out by spin coating, sputtering or vacuum vapour deposition. The phthalocyanines of the formulae (Ia) to (Ih) can be applied particularly well by vacuum vapour deposition or sputtering, in particular vacuum vapour deposition.

Starting materials for such coatings applied by sputtering or vacuum vapour deposition are all the abovementioned forms of the phthalocyanines of the formula (I), i.e. fine powders, finely crystalline forms or granular materials, particulate solid preparations, solid shaped bodies and dispersions. The latter are employed particularly for applying the phthalocyanines in finely divided form to a surface from which they can then be applied to the substrate by sputtering or vacuum vapour deposition.

Phthalocyanine purities of greater than 50%, particularly preferably greater than 85% and very particularly preferably greater than 90%, in particular greater than 95% or greater than 98%, are preferred for these procedures.

The phthalocyanines can be mixed with one another or with other dyes having similar spectral properties. The information layer can comprise not only the phthalocyanines but also additives such as binders, wetting agents, stabilizers, diluents and sensitizers and also further constituents.

The invention further provides an apparatus for the vapour deposition of light-absorbent compounds onto a substrate for producing optical storage media, which is characterized in that the dye can be vaporized by heating at a low background pressure and be deposited on the substrate. The background pressure is below 10⁻¹ Pa, preferably below 10⁻³ Pa, particularly preferably below 10⁻⁴ Pa. The dye is preferably heated by means of resistive heating or by microwave absorption.

In particular, the invention provides an optical data carrier as described above in which the light-absorbent compound of the formula (I), if appropriate together with the abovementioned additives, forms an information layer which is optically amorphous. For the purposes of the present invention, amorphous means that no crystallites can be observed under an optical microscope and no Bragg reflections but only an amorphous halo can be observed in the X-ray diffraction pattern.

Apart from the information layer, further layers such as metal layers, dielectric layers and protective layers can be present in the optical data store. Metals and dielectric layers serve, inter alia, to adjust the reflectivity and the heat absorption/retention. Metals can be, depending on the laser wavelength, gold, silver, aluminium, alloys, etc. Examples of dielectric layers are silicon dioxide and silicon nitride. Protective layers are, for example, photocurable surface coatings, adhesive layers and protective films.

The adhesive layers can be pressure-sensitive.

Presssure-sensitive adhesive layers consist mainly of acrylic adhesives. Nitto Denko DA-8320 or DA-8310, disclosed in the patent JP-A 11-273147, can, for example, be used for this purpose.

The optical data carrier has, for example, the following layer structure (cf. FIG. 1): a transparent substrate (1), if desired a protective layer (2), an information layer (3), if desired a protective layer (4), if desired an adhesive layer (5), a covering layer (6).

The structure of the optical data carrier preferably:
comprises a preferably transparent substrate (1) to whose surface at least one light-writable information layer (3) which can be written on by means of light, preferably laser light, if desired a protective layer (4), if desired an adhesive layer (5) and a transparent covering layer (6) have been applied.
comprises a preferably transparent substrate (1) to whose surface a protective layer (2), at least one information layer (3) which can be written on by means of light, preferably laser light, if desired an adhesive layer (5) and a transparent covering layer (6) have been applied.
comprises a preferably transparent substrate (1) to whose surface a protective layer (2) if desired, at least one information layer (3) which can be written on by means of light, preferably laser light, if desired a protective layer (4), if desired an adhesive layer (5) and a transparent covering layer (6) have been applied.
comprises a preferably transparent substrate (1) to whose surface at least one information layer which can be written on by means of light, preferably laser light, if desired an adhesive layer (5) and a transparent covering layer (6) have been applied.

Alternatively, the optical data carrier has, for example, the following layer structure (cf. FIG. 2): a preferably transparent substrate (11), an information layer (12), if desired a reflection layer (13), if desired an adhesive layer (14), a further preferably transparent substrate (15).

Alternatively, the optical data carrier has, for example, the following layer structure (cf. FIG. 3): a preferably transparent substrate (21), an information layer (22), if desired a reflection layer (23), a protective layer (24).

Alternatively, the structure of the optical data carrier can
comprise a plurality of information layers which are preferably separated by suitable layers. Particularly preferred separating layers are photocurable resins, adhesive layers, dielectric layers or reflection layers.

The invention further provides optical data carriers according to the invention which have been written on by means of blue light, in particular laser light, particularly preferably laser light having a wavelength of 360-460 mm.

The following examples illustrate the invention.

### EXAMPLES

### Example 1

a) 4.75 g of 2-methyl-3,4-diethylpyrrole-5-t-butylcarboxylate and 6.73 g of potassium hydroxide powder were suspended in 40 ml of dimethyl sulphoxide and stirred for one hour. 3.42 g of benzyl bromide were subsequently added dropwise and the solution was firstly stirred at RT for another one hour and then at 70° C. for 30 minutes. The suspension was diluted with 50 ml of water and the product of the formula 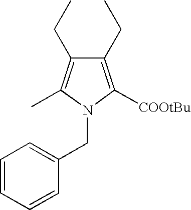
   which had precipitated as a white powder was filtered off and washed with water. Drying gave 5.5 g (84% of theory) of product. M.p.=75-77° C.
b) 5.43 g of the compound from a) and 0.80 g of 3,4-dihydroxy-3-cyclobutene-1,2-dione in 20 ml of ethanol were admixed with 0.5 ml of 37% strength hydrochloric acid and the mixture was refluxed for 4 hours. After taking off the solvent, the residue was digested in diethyl ether and the solid which precipitated was filtered off and washed with diethyl ether. Drying gave 2.5 g (47% of theory) of green powder of the formula 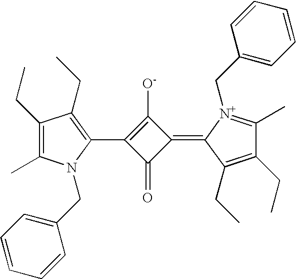
m.p.=203° C. (decomposition)
molecular mass=532.73
λₘₐₓ=590 nm (dichloromethane)
ε=167 000 l/mol cm λ_{1/2}−λ_{1/10} (long wavelength flank)=21 nm Δλ=λ_{DMF}−λ_{dioxane}|=2 nm

### Example 2

a) 4.19 g of 2-methyl-3,4-diethylpyrrole-5-ethylcarboxylate and 3.37 g of potassium hydroxide powder in 30 ml of dimethyl sulphoxide were stirred at 80° C. for 10 minutes. After cooling, 6.14 g of 3,5-bistrifluoromethylbenzyl bromide were added dropwise and the solution was stirred at RT for another one hour and subsequently at 70° C. for two hours. The solution was diluted with 200 ml of water, extracted with dichloromethane and the product was precipitated by acidification with hydrochloric acid. Filtration and washing with water gives, after drying, 4.29 g (53% of theory) of product of the formula 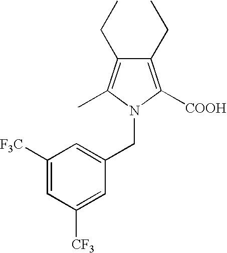
   in the form of a colourless powder. M.p.=126-128° C.
b) Use of 4.07 g of the pyrrole compound from a) and 0.57 g of 3,4-dihydroxy-3-cyclobutene-1,2-dione in a procedure analogous to that of Example 1b gave 3.29 g (82% of theory) of a green powder of the formula 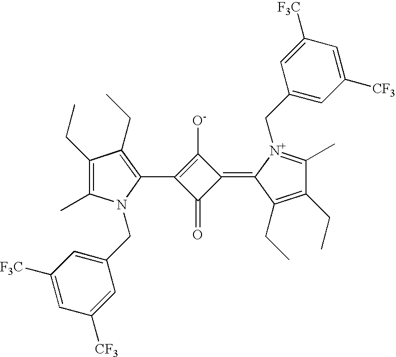
m.p.=185° C.
molecular mass=804.73
λₘₐₓ=590 nm (dichloromethane)
ε=184 652 l/mol cm
λ_{1/2}−λ_{1/10} (long wavelength flank)=19 nm

### Example 3

a) Using a method analogous to Example 2a, 1.90 g of 2,4-dimethylpyrrole, 2.24 g of potassium hydroxide powder and 4.78 g of 4-trifluoromethylbenzyl bromide were reacted in 20 ml of dimethyl sulphoxide. After the reaction was complete, the solution was diluted with 300 ml of water and the product was extracted with dichloromethane. The organic phase was dried over Na₂SO₄ and filtered. Taking off the solvent gave 4.8 g (95% of theory) of product as a brown resin of the formula 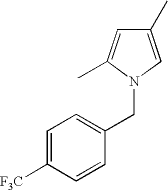
   1.77 g of the product from a) and 0.4 g of 3,4-dihydroxy-3-cyclobutene-1,2-dione were reacted in a procedure similar to that of Example 1b. After cooling, the product was precipitated in 400 ml of diisopropyl ether and filtered off to give 0.2 g of the product

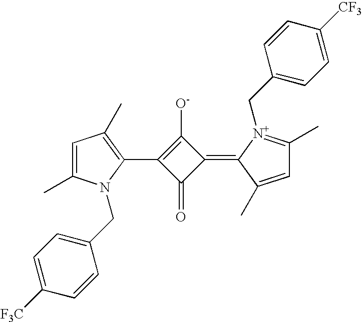

The filtrate was evaporated and chromatographed on silica using dichloromethane as eluant. The first violet fraction gave, after taking off the solvent, a further 0.72 g of product. Total yield: 0.92 g (45% of theory).
m.p.=220° C.
molecular mass=584.57 λₘₐₓ=577 nm (acetone)
ε=139 000 l/mol cm λ_{1/2}−λ_{1/10} (long wavelength flank)=20 nm

### Example 4

a) 4.19 g of 2-methyl-3,4-diethylpyrrole-5-ethylcarboxylate and 3.37 g of potassium hydroxide powder in 20 ml of dimethyl sulphoxide were stirred at 80° C. for 10 minutes. After cooling, 4.78 g of 2-trifluoromethylbenzyl bromide were added dropwise and the solution was stirred at RT for another one hour and subsequently at 70° C. for three hours. The solution was diluted with 300 ml of water, extracted with dichloromethane and the product was precipitated by acidification with hydrochloric acid. Filtration and washing with water gives, after drying, 3.80 g (56% of theory) of product of the formula 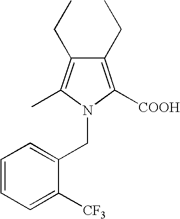
   in the form of a colourless powder. M.p.=124-126° C.
b) 3.39 g of the compound from a) and 0.57 g of 3,4-dihydroxy-3-cyclobutene-1,2-dione in 20 ml of ethanol were admixed with 0.5 ml of 37% strength hydrochloric acid and the mixture was refluxed for 4 hours. After cooling, the solid which had precipitated was filtered off and washed with diisopropyl ether. Drying gave 2.58 g (77% of theory) of green powder of the formula 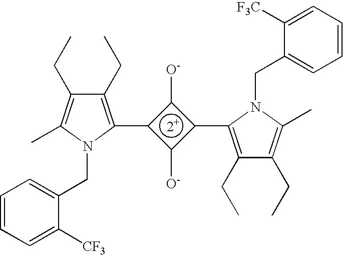
m.p.=206° C.
molecular mass=668.73
λₘₐₓ=588 nm (acetone)
ε=202 000 l/mol cm λ_{1/2}−λ_{1/10} (long wavelength flank)=22 nm
Δλ=|λ_{DMF}−λ_{dioxane}|=1 nm

Further suitable squarylium dyes are shown in the table. These are obtained by analogous preparation of the components and the squarylium dyes.

| Example | R¹ | R² | R³ | R⁴ | λₘₐₓ/nm¹⁾ | ε/1/mol cm | λ_{1/2}-λ_{1/10}/nm²⁾ | Δλ³⁾ |
|---|---|---|---|---|---|---|---|---|
| 1 | 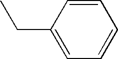 | CH₃ | C₂H₅ | C₂H₅ | 590 | 167 000 | 21 | 2 |
| 2 | 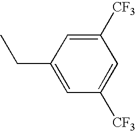 | CH₃ | C₂H₅ | C₂H₅ | 590 | 184652 | 19 | |
| 3 | 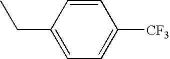 | CH₃ | H | CH₃ | 577 | 139 000 | 20 | |
| 4 | 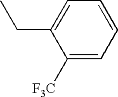 | CH₃ | C₂H₅ | C₂H₅ | 588 | 202 000 | 22 | 1 |
| 4a | 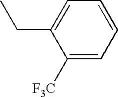 | CH₃ | C₂H₅ | Ph-C₆H₅ | | | | |
| 4b | 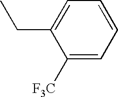 | CH₃ | COOC₂H₅ | CH₃ | 583 | | | |
| 5 | C₂H₅ | CH₃ | C₂H₅ | C₂H₅ | 587 | | | |
| 6 | C₅H₁₁ | CH₃ | C₂H₅ | C₂H₅ | 588 | 189 000 | 21 | |
| 7 | 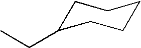 | CH₃ | C₂H₅ | C₂H₅ | 587 | 184 000 | 20 | |
| 8 | 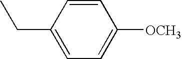 | CH₃ | C₂H₅ | C₂H₅ | 591 | 198 000 | 21 | |
| 9 | 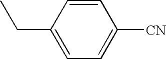 | CH₃ | C₂H₅ | C₂H₅ | 589 | 148 000 | 20 | |
| 9a | 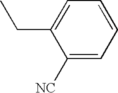 | CH₃ | C₂H₅ | C₂H₅ | 589 | | | |
| 10 | 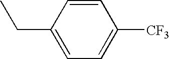 | CH₃ | H | C₂H₅ | 578 | | | |
| 11 | 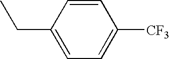 | CH₃ | C₂H₅ | C₂H₅ | 588 | 187 000 | 18 | 1 |
| 12 | 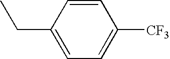 | CH₃ | COCH₃ | C₂H₅ | 578 | | | |
| 13 | 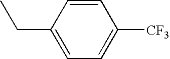 | CH₃ | COOC₂H₅ | CH₃ | 577 | | | |
| 14 | 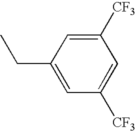 | CH₃ | H | CH₃ | 591 | 185 000 | 19 | |
| 15 | 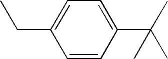 | CH₃ | C₂H₅ | C₂H₅ | 589 | 146 000 | | 1 |
| 16 | 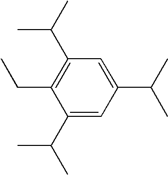 | CH₃ | C₂H₅ | C₂H₅ | 599 | 149 000 | 26 | 0 |
| 17 | 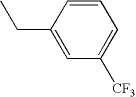 | CH₃ | COCH₃ | C₂H₅ | 605 | | | |
| 17a | 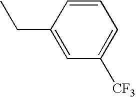 | CH₃ | COCH₃ | C₂H₅ | 578 | | | |
| 18 | 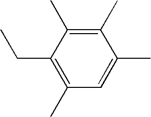 | CH₃ | C₂H₅ | C₂H₅ | 595 | | | |
| 19 | H | CH₃ | C₂H₅ | C₂H₅ | 568 | | | |
| 19a | H | CH₃ | COOC₂H₅ | CH₃ | 563 | | | |

¹⁾in acetone unless indicated otherwise
²⁾on the long wavelength flank
³⁾Δλ = |λ_{DMF} − λ_{dioxane}|

### Example 20

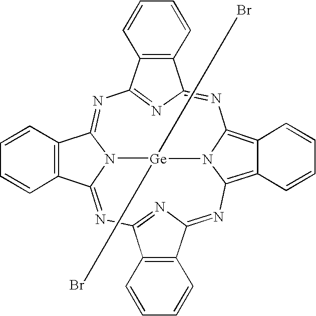 The dye dibromogermanium phthalocyanine (GeBr₂Pc) was vapour-deposited in a high vacuum (pressure p 2-10⁻⁵ mbar) from a resistively heated molybdenum boat onto a pregrooved polycarbonate substrate at a rate of about 5 A/s. The layer thickness was about 55 nm. The pregrooved polycarbonate substrate had been produced as a disk by means of injection moulding. The diameter of the disk was 120 mm and its thickness was 0.6 mm. The groove structure produced in the injection moulding process had a track spacing of about 1 μm and the groove depth and groove width at half height were about 150 nm and about 260 nm, respectively. The disk with the dye layer as information carrier was coated with 100 nm of Ag by vapour deposition. A UV-curable acrylic coating was subsequently applied by spin coating and cured by means of a UV lamp. The disk was tested by means of a dynamic writing test apparatus constructed on an optical test bench comprising a GaN diode laser (λ=405 nm) for generating linearly polarized laser light, a polarization-sensitive beam splitter, a A/4 plate and a movably suspended collecting lens having a numerical aperture NA=0.65 (actuator lens). The light reflected from the disk was taken out from the beam path by means of the abovementioned polarization-sensitive beam splitter and focused by means of an astigmatic lens onto a four-quadrant detector. At a linear velocity V=5.00 m/s and a writing power P_{w}=13 mW, a signal/noise ratio C/N=41 dB was measured. The writing power was applied as a pulse sequence, with the disk being irradiated alternatively for 1 μs with the abovementioned writing power P_{w} and for 4 μs with the reading power Pᵣ=0.44 mW. The disk was irradiated with this pulse sequence until it had rotated once. The markings produced in this way were then read using the reading power Pᵣ=0.44 mW and the abovementioned signal/noise ratio CIN was measured.

### Example 21

9.95 g of the dichlorotin phthalocyanine of the formula 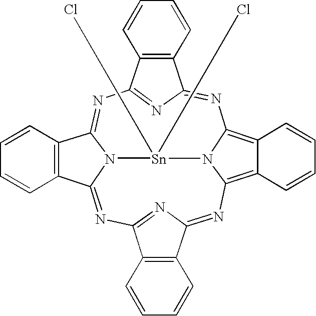
in 125 ml of pyridine were admixed at room temperature with 4.9 g of sodium tetrahydroborate. The mixture was stirred under reflux (115° C.) for 75 minutes and slowly cooled to 90° C. At this temperature, 100 ml of water were slowly added dropwise. The mixture was then refluxed for another 30 minutes, cooled to room temperature and filtered with suction. The filter cake was stirred with 200 ml of methanol, filtered with suction and washed with methanol until the washings were clear. After washing with 50 ml of water, it was dried at 30° C. under reduced pressure. This gave 4.03 g (63% of theory) of a blue powder of the formula 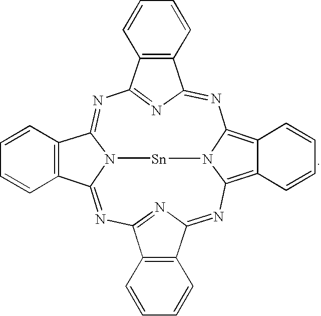
1.6 g of this product in 32 ml of chloronaphthalene which had been dried over molecular sieves were admixed at room temperature with a solution of 0.2 ml of bromine in 10 ml of chloronaphthalene while stirring. The mixture was then stirred at 70-75° C. for one hour, with the temperature rising briefly to 90° C. After cooling to room temperature, the mixture was filtered with suction, washed with toluene until the washings were clear, subsequently washed with methanol until the washings were virtually colourless and dried at 30° C. under reduced pressure. This gave 1.46 g (74% of theory) of a blue powder of the formula

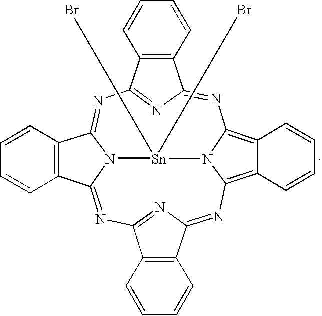

1. Squarylium compounds of the formula I 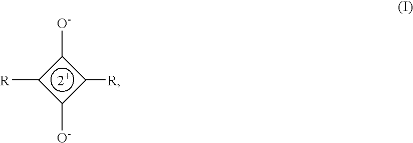
where
R is a heterocyclic five-membered ring, with the exception of amino-substituted furan rings.

2. Compounds according to claim, 1, characterized in that R is a substituted or unsubstituted pyrrole.

3. Compounds according to claim 1, characterized in that they have the formula Ia 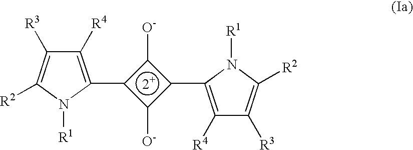
where
R¹ is hydrogen, substituted or unsubstituted alkyl or substituted or unsubstituted aralkyl,
R² is substituted or unsubstituted alkyl, substituted or unsubstituted aryl, alkoxycarbonyl or substituted or unsubstituted alkylcarbonyl, and
R³ and R⁴ are each, independently of one another, hydrogen, substituted or unsubstituted alkyl, substituted or unsubstituted aryl, alkoxycarbonyl or substituted or unsubstituted alkylcarbonyl.

4. Compounds according to claim 3, characterized in that
R¹ is hydrogen, methyl, ethyl, propyl, butyl, cyclohexylmethyl, benzyl, 4-methoxybenzyl, 4-trifluoromethylbenzyl, 3-trifluoromethylbenzyl, 2-trifluoromethylbenzyl, 3,5-bis(trifluoromethyl)benzyl or 4-fluoro-2-trifluoromethylbenzyl,
R² is methyl, ethyl, propyl or phenyl and
R³ and R⁴ are each, independently of one another, hydrogen, methyl, ethyl propyl, butyl, phenyl, acetyl or ethoxycarbonyl.

5. Compounds according to claim 3, characterized in that
R¹ is 4-trifluoromethylbenzyl or 3,5-bis(trifluoromethyl)benzyl, in particular 4-trifluoromethylbenzyl,
R² is methyl, ethyl or phenyl, in particular methyl,
R³ is hydrogen, ethyl, acetyl or ethoxycarbonyl, in particular ethyl, and
R⁴ is methyl, ethyl or phenyl, in particular methyl or ethyl.

6. Process for preparing the squarylium compounds according to claim 1, which is characterized in that 3,4-dihydroxy-3-cyclobutene-1,2-dione (squaric acid) is reacted with at least one compound of the formula III
R—R⁵ (III),
in particular with a pyrrole compound of the formula (IIIa), 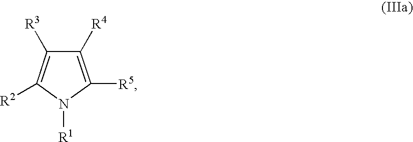
where
R⁵ is hydrogen, ethoxycarbonyl or carboxyl and
R¹ to R⁴ are as defined above.

7. Use of squarylium compounds according to claim 1 as light-absorbent compounds in the information layer of write-once optical data carriers.

8. Use according to claim 7, characterized in that the optical data carrier can be written and read by means of red laser light, in particular having a wavelength in the range 600-680 nm.

9. Pyrroles of the formula (IIIa) 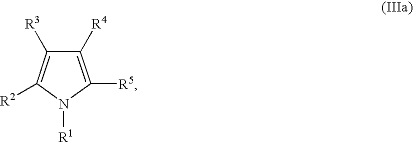
where
R¹ is substituted or unsubstituted C₃-C₁₂-alkyl or substituted or unsubstituted aralkyl,
R² is substituted or unsubstituted alkyl, substituted or unsubstituted aryl, alkoxycarbonyl, carbonyl or substituted or unsubstituted alkylcarbonyl,
R³ and R⁴ are each, independently of one another, hydrogen, substituted or unsubstituted alkyl, substituted or unsubstituted aryl, alkoxycarbonyl, carbonyl or substituted or unsubstituted alkoxycarbonyl and
R⁵ is hydrogen, alkoxycarbonyl or carboxyl.

10. Pyrrole compounds according to claim 9, characterized in that
R¹ is propyl, butyl, cyclohexylmethyl, benzyl, 4-methoxybenzyl, 4-trifluoromethylbenzyl, 3-trifluoromethylbenzyl, 2-trifluoromethylbenzyl, 3,5-bis(trifluoromethyl)benzyl or 4-fluoro-2-trifluoromethylbenzyl,
R² is methyl, ethyl, propyl or phenyl,
R³ and R⁴ are, independently of one another, hydrogen, methyl, ethyl, propyl, butyl, phenyl, acetyl or ethoxycarbonyl and
R⁵ is hydrogen, t-butoxycarbonyl or carboxyl.

11. Process for preparing pyrroles of the formula (11a) according to claim 9, characterized in that a pyrrole compound of the formula (II) 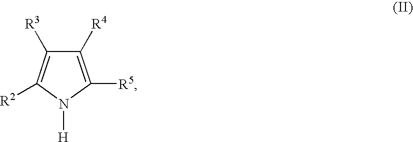
where
R² to R⁴ are as defined in claim 9 for the pyrroles of the formula IIIa and
R⁵ is hydrogen, alkoxycarbonyl, in particular t-butoxycarbonyl or ethoxycarbonyl, or carboxyl,
is reacted with a halogen compound of the formula IV
R¹—X (IV),
where
R¹ is as defined in claim 9 for the pyrroles of the formula IIIa,
X is Cl, Br or 1,
and at least 2 equivalents of a base.

12. Optical data carrier comprising a preferably transparent substrate to whose surface a light-writable information layer, if desired one or more reflection layers and a further substrate or a protective layer have been applied, which can be written on or read by means of red light, preferably laser light, where the information layer comprises a light-absorbent compound, characterized in that at least one squarylium compound according to at least one of claims 1 to 5 is used as light-absorbent compound.

13. Process for producing the optical data carriers according to claim 12, which is characterized in that a preferably transparent substrate is coated with squarylium compounds according to claim 1, if desired in combination with suitable binders and additives and if desired suitable solvents and is, if desired, provided with a reflection layer, further intermediate layers and, if desired, a protective layer or a further substrate.

14. Optical data carriers according to claim 12 which have been written on by means of red light, in particular red laser light.

15. Optical data carrier comprising a preferably transparent substrate which may, if desired, have previously been coated with one or more reflection layers and to whose surface a light-writable information layer, if desired one or more reflection layers and if desired a protective layer or a further substrate or a covering layer have been applied, which can be written on or read by means of blue light, preferably laser light, particularly preferably light having a wavelength of 360-460 nm, in particular 380-420 nm, very particularly preferably 390-410 nm, or by means of infrared light, preferably laser light, particularly preferably light having a wavelength of 760-830 nm, where the information layer comprises a light-absorbent compound and, if desired, a binder, characterized in that at least one phthalocyanine of the formula (I) 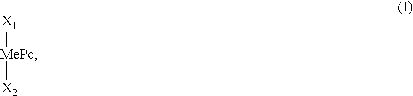
where
Me is a doubly axially substituted metal atom from the group consisting of Si, Ge and Sn,
Pc is an unsubstituted phthalocyanine and
X¹ and X² are each, independently of one another, bromine or iodine and X¹ may also be chlorine,
is used as light-absorbent compound.

16. Optical data store according to claim 1, characterized in that at least one phthalocyanine of the formulae (Ia), (Ib), (Ic), (Id), (Ie), (If), (Ig) and (Ih) 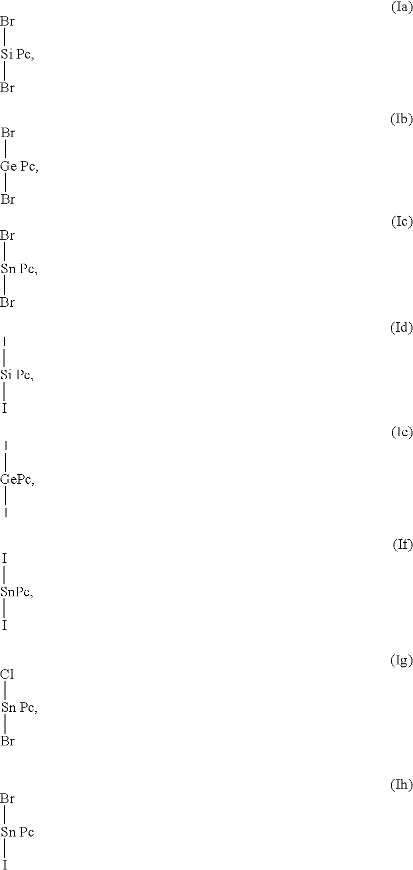
is used as light-absorbent compound.

17. Use of phthalocyanines of the formula (I) 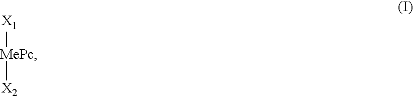
where
Me is a doubly axially substituted metal atom from the group consisting of Si, Ge and Sn,
Pc is an unsubstituted phthalocyanine and
X¹ and X² are each, independently of one another, bromine or iodine and X¹ may also be chlorine,
as light-absorbent compound in the information layer of optical storage media.

18. Use of phthalocyanines 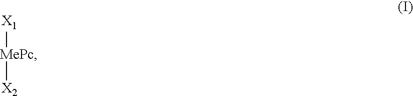
where
Me is a doubly axially substituted metal atom from the group consisting of Si, Ge and Sn,
Pc is an unsubstituted phthalocyanine and
X¹ and X² are each, independently of one another, bromine or iodine and X¹ may also be chlorine,
for producing optical storage media.

19. Use according to claim 17, characterized in that the phthalocyanines used have a purity of more than 90% by weight, in particular more than 95% by weight, particularly preferably more than 98% by weight, based on the phthalocyanine of the formula (I).

20. Process for coating substrates with the phthalocyanines of the formula (I) 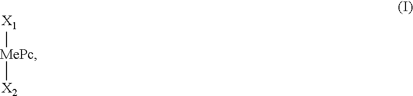
where
Me is a doubly axially substituted metal atom from the group consisting of Si, Ge and Sn,
Pc is an unsubstituted phthalocyanine and
X¹ and X² are each, independently of one another, bromine or iodine and X¹ may also be chlorine.

21. Optical data carriers according to claim 15 which have been written on by means of blue light, in particular laser light, particularly preferably laser light having a wavelength of 360-460 nm.
